(19) United States
(12) Patent Application Publication
(10) Pub. No.: US 2008/0226579 A1
(43) Pub. Date: Sep. 18, 2008

(54) Novel Resinate Complex of S-Clopidogrel and Production Method Thereof

(75) Inventors: Hee Jong Shin, Gyeonggi-do (KR); Min Hyo Ki, Chungcheongnam-do (KR); Mee Hwa Choi, Chungcheongnam-do (KR)

Correspondence Address: SUGHRUE MION, PLLC, 2100 PENNSYLVANIA AVENUE, N.W., SUITE 800, WASHINGTON, DC, 20037, US

(73) Assignee: Chong Kun Dang Pharmaceutical Corp., Seoul (KR)

(21) Appl. No.: 12/065,104

(22) PCT Filed: Aug. 24, 2006
(86) PCT No.: PCT/KR06/03346

§ 371 Date: Feb. 28, 2008

(30) Foreign Application Priority Data
Sep. 21, 2005 (KR) 10-2005-0087628

(51) Int. Cl. A61K 47/48; C07D 495/04; A61P 7/00

(52) U.S. Cl. 424/78.1; 521/25

(57) ABSTRACT

The present invention is a novel resinate complex of (+)-clopidogrel optical isomer, wherein the (+)-clopidogrel isomer is bounded to a water-soluble cation exchange resin having sulfonic acid groups. The novel resinate complex has recognized some advantages in that (1) its chemical structure is stable, and (2) it can be formulated into a solid form that may provide taste-masking capabilities associated with bitter drugs (e.g., strong irritation, bitterness and sour taste), thus requiring no drink of water.

### TECHNICAL FIELD

The present invention is a novel resinate complex of (+)-clopidogrel optical isomer, wherein the (+)-clopidogrel isomer is bounded to a water-soluble cation exchange resin having sulfonic acid groups.

### BACKGROUND ART

Clopidogrel has shown to have activity with inhibitory properties towards platelet aggregation, and is useful for the treatment and prevention of thromboembolism such as stroke or myocardial infarction. Dextrorotary clopidogrel or racemate exhibits activity on platelet aggregation, whereas the levorotatory isomer is less active and poorly tolerated.

Clopidogrel (free base) is a semi-solid (oily) form with high-viscosity flowability, affecting the storage or handling process. Its low solubility to water makes it difficult to industrially develop a pharmaceutical product.

In order for clopidogrel to be administered to human body as a medicine, it should necessarily take a solid form (preferably powder) which may be dissolved in water.

Processes for manufacturing crystallized form of clopidogrel (free base) have been disclosed in the prior arts.

The Korean Patent Publication No. 1987-1270 disclosed a variety of acidic salts (hydrochloric acid, sulfuric acid) which aims to crystallize an oily clopidogrel racemate. The Korean Patent Publication No. 1996-3615 described a method of crystallizing an oily (+)-clopidogrel isomer using some water-soluble salts (sulfate, taurocholate, and bromate) that serves to easily achieve the crystallization of active clopidogrel with no hygroscopicity.

WO 04/106344, WO 05/016931, U.S. Pat. No. 4,847,265, U.S. Patent Application No. 2004/0132765 and U.S. Patent Application No. 2005/0059696 disclosed the solid forms of monomolecular clopidogrel addition acidic salts as a single molecule. The Korean Patent Unexamined Publication No. 2005-8692A disclosed clopidogrel sulfonate.

The prior art processes focus on the method for preparing crystallized clopidogrel by binding clopidogrel (free base) with monomolecular acidic materials, but fail to disclose the fact that a complex in powder form may be prepared by binding an unstable oily clopidogrel (free base) to a cation exchange resin, as demonstrated in the present invention.

Clopidogrel (free base) has a crystalline or amorphous solid form in the concentrated inorganic or organic acid. If the use of monomolecular acidic materials employed for making the clopidogrel addition salts, ester in clopidogrel (free base) is hydrolyzed into carboxylic acid and methanol (An ester compound is decomposed by an acid, thus generating acid and alcohol). The carboxylic acid compound thus formed is an impurity of clopidogrel.

The monomolecular acidic materials used for preparing clopidogrel addition salts affects the stability of ester in the molecule, thus making its structure unstable before the drug is delivered to the body and affecting the safety and efficacy of clopidogrel in the gastrointestinal tract.

The use of a cation exchange resin bounded to a (+)-clopidogrel isomer according to the present invention is different from that of the conventional pharmaceutical and chemical fields, where ion exchange resins have been used for many years in pharmaceutical formulation and their use have ranged from simple excipients for tablet disintegration to the rate controlling function in extended release formulation.

WO 03/051362, U.S. Pat. No. 6,767,913, U.S. Patent Application No. 2003/0114479, U.S. Patent Application No. 2005/0049275, U.S. Patent Application No. 2005/0008702 and the Korean Patent Unexamined Publication No. 2004-66917A disclosed the use of cation exchange resin as a disintegrant to increase the rate of dissolution.

WO 99/30690, WO 04/103349 and U.S. Pat. No. 6,800,668 disclosed the use of an ion exchange resin as a sustained release agent or stabilizer.

Meantime, sustained release formulations containing a complex of a cation exchange resin and pharmaceutical compounds are disclosed in the prior arts.

U.S. Pat. No. 2,990,332 disclosed a process for preparing a drug loaded onto water-insoluble ion exchange resin with a particle size of about 500□ or less for controlled release of the drug for at least 8 hours.

U.S. Pat. No. 3,608,063 disclosed a process for preparing polymer particles that may be released from latex in water.

U.S. Pat. No. 4,369,175 disclosed a process for manufacturing sustained release vicamine resinate directly from the resinate of alkali metal or alkaline earth metal salt.

U.S. Pat. No. 4,788,055 disclosed a resinate sustained release dextromethorphan composition.

U.S. Pat. Nos. 3,138,525, 4,762,709 and 4,996,047 disclosed a process for preparing a sustained release agent, wherein polymer particles are prepared with or without an additive to support the incomplete sustained release property of an agent-resin complex, followed by coating the complex with a water-permeable diffusion barrier (water-soluble or water-insoluble).

In the prior art processes, all resin-drug complexes refer to achievement of different types of modified release with water-insoluble ion exchange resins. In contrast, the novel resinate complex of (+)-clopidogrel isomer according to the present invention, where an unstable (+)-clopidogrel isomer is bound with a water-soluble cation exchange resin, has better stability in a chemical structure, thus improving its formulation stability and dissolution rate as well.

U.S. Pat. No. 5,980,882 disclosed a pharmaceutical composition comprising a drug-resin complex and a chelating agent (EDTA) for improving the stability of a pharmaceutical composition.

U.S. Pat. Nos. 4,459,278 and 5,643,560 disclosed the use of ion exchange resins to modify drug release rate, drug absorption and reduce side effects, while sustaining a plasma concentration.

Nevertheless, the prior art processes are not related to the present invention in that the clopidogrel-resinate complex of the present invention is not stabilized by EDTA and is not associated with side effects by its absorption into the body.

Additionally, clopidogrel is a drug which is unpleasant to take orally in the mouth and/or gastrointestinal tract due to a strong irritation, bitter and sour taste.

A composition comprising the drug-resin complexes of the present invention surprisingly has a taste-masking capabilities (strong irritation, bitterness and obnoxious taste) against (+)-clopidogrel sulfate.

The Korean Patent Unexamined Publication No. 2004-66917 disclosed the use of a viscosity enhancer in clopidogrel hydrogen sulfate for a pleasant tasting with good mouth-feel. This taste masking has been limited with little improvement of bitter and obnoxious taste.

In the prior art processes, the use of ion exchange resins are disclosed for taste masking.

U.S. Pat. No. 3,901,248 disclosed a chewable smoking substitute composition that comprises a chewing gum base and nicotine in combination with certain saliva-insoluble cation exchange resins.

U.S. Pat. Nos. 5,032,393 and 5,219,563 disclosed a chewing gum or sucking agent by adsorption of ranitidine onto the ion exchange resin particles to form the drug-resin complex.

U.S. Pat. No. 6,514,492 disclosed a pharmaceutical suspension composition comprising a quinolone antibiotic, one or more expients and an ion exchange resin.

As taste masking occurs due to intrinsic nature of drug molecules, the prior art processes is not related to the masking effect of strong irritation in the mucosa and taste change that may be induced by the clopidogrel-resinate complex of the present invention. As the prior art processes aiming to prepare liquid compositions are characterized by water-insoluble ion exchange resins for taste masking effect, this is entirely different from the drug-resin complex of the present invention using the water-soluble ion resin causing tastelessness or no bitterness in aqueous media.

The use of some acidic materials for crystallized (+)-clopidogrel isomer in the prior art may affect the stability of ester in the clopidogrel molecules and thus the inventors have focused on the formulation study to ensure better safety and efficacy of drug.

The present invention is based on the finding that the resinate complex of (+)-clopidogrel isomer bounded with water-soluble cation exchange resin had better stability in its chemical structure, taste-masking capabilities (strong irritation, bitterness and obnoxious taste) against(+)-clopidogrel sulfate and a variety of dosage forms, such as solid preparation requiring no drink of water and liquid preparation (syrup).

### DISCLOSURE OF INVENTION

### Technical Problem

It is an object of the present invention to provide a novel clopidogrel bulk material whose crystal structure is stable.

A further object of the present invention is to provide a novel clopidogrel bulk material with a combination of hygroscopicity-free property and excellent flowability to a powder form, and as a result, such bulk material can be easily formulated into particular dosage forms.

Another object of the present invention is to provide a novel clopidogrel bulk material that may be formulated as a solid preparation with no drink of water or a liquid preparation (e.g., syrup) by taste-masking capabilities (strong irritation, bitterness and obnoxious taste) against hydrogen sulfate of (+)-clopidogrel isomer.

Another object of the present invention is to provide a process for manufacturing a novel clopidogrel bulk material.

### Technical Solution

To achieve the aforementioned objective, the present invention is to provide a clopidogrel-resinate complex and its manufacturing method, wherein an oily (+)-clopidogrel isomer (free base) is bounded to a water-soluble cation exchange resin having sulfonic acid groups and a molecular weight range of 5,000 to 1,000,000. The clopidogrel-resinate complex is characterized by the fact that (1) its crystal structure is stable, and (2) it provide excellent flowability to a powder form.

Clopidogrel of the present invention is a generic name for methyl(+)-(S)-(2-chlorophenyl)(6,7-dihydro-4H-thieno[3,2-c]pyridine-5-yl)acetate as a dextrorotary optical isomer.

Also, polymers used in the clopidogrel-resinate complex of the present invention represent styrene sulfonate polymer or divinylbenzene styrene sulfonate copolymer.

The water-soluble cation exchange resin having sulfonic acid groups of the present invention is characterized by an ion exchange capacity.

In particular, such resin has sulfonic acid-derived anionic groups that can be reacted with amine groups of the drug. Preferably, the molecular weight of a water-soluble polymer is in the range from 5,000 to 1,000,000. More preferably, the molecular weight of a water-soluble polymer is in the range from 10,000 to 500,000. If the molecular weight of a water-soluble polymer exceeds 1,000,000, its solubility loss may occur. As a result, the water-insoluble cation exchange resin having sulfonic acid groups has a limit in binding clopidogrel to resin particles, requiring excess amount of ion exchange resins. The final solid product does not contribute to the structural stability of (+)-clopidogrel isomer.

A water-soluble polymer used for forming the clopidogrel-resinate complex may be selected from a cation exchange resin forming as styrene, or styrene with divinyl benzene, as shown in the following formula 1 or 2.

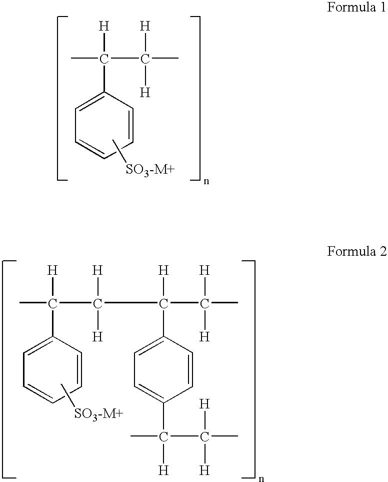

Where, [M⁺] is a substituted acidic sulfonic acid in the benzene structure but is further replaced by hydrogen atom or acid derivatives having a variety of substituted alkali metal or alkali earth metal. In the case of a polymer substituted into metal atom, the polymer is pretreated with a strong acid to recover an ionic binding capacity and washed with water or organic solvent to form a complex of clopidogrel (free base) with resin.

More specifically, the ion exchange resins suitable for use in the present invention may be one or more polymers selected from the group consisting of water-soluble styrene sulfonate polymer and divinyl benzene styrene sulfonate copolymer.

The ion exchange resins bounded to alkali metal or alkaline earth metal (e.g., sodium styrene sulfonate polymer, potassium styrene sulfonate polymer, calcium styrene sulfonate polymer, sodium divinyl benzene styrene sulfonate copolymer, potassium divinyl benzene styrene sulfonate copolymer and calcium divinyl benzene styrene sulfonate copolymer) do not deviate the scope of the present invention.

In order to prepare the clopidogrel-resinate complex of the present invention, the present invention provides an oily (+)-clopidogrel isomer (free base) that is a sticky amorphous semi-solid material in transparent, colorless or weakly brown color at room temperature. Its solubility to water is extremely low. Also, the clopidogrel-resinate complex of the present invention is prepared by removing the salts from clopidogrel salts and substituting ion exchange resins.

The present invention relates to a process for preparing the clopidogrel-resinate complex whose chemical structure is stable until the complex is delivered into a target site of the body from the gastrointestinal tract, while preventing the possible occurrence of impurities. The clopidogrel-resinate complex is prepared in an easy, stable manner, and its formulation into a variety of dosage forms is also available on an industrial basis. The drug-resinate complex, so formed, is not hygroscopic in the air and provide excellent flowability to the powder form. The solid-form complex starts to dissolve or decompose at more than 200° C.

The clopidogrel-resinate complex according to the present invention, so formed from the cation exchange resin, is a novel drug which contributes to better stability of clopidogrel that is unstable in free base. Its solid or powder form makes it easier for the novel drug-resin complex to be used for a large industrial production. Those skilled in the art may appreciate that unlike other organic or inorganic acid salts, the cation exchange resin having sulfonic acid groups according to the present invention serves to deliver clopidogrel only to the gastrointestinal tract by avoiding its absorption in the body, thus ensuring better safety profile.

The (+)-clopidogrel isomer-resinate complex according to the present invention is obtained as a solid form in aqueous media containing an organic solvent or water. An oily (+)-clopidogrel isomer (free base) is homogenously pasted or dissolved in the organic solvent used in the methodology of the present invention. Examples of solvents include one or more solvents selected from the group consisting of organic solvent and water. The preferred solvent may include one or more anhydrous organic solvents. More preferred solvents useful in the present invention may include, but are not limited to one or more solvents selected from the group consisting of acetone, methanol, ethanol, diethyl ether, diisopropyl ether, t-butylmethyl ether and hexane.

A solid form is precipitated such that the solution of a cation exchange resin is added dropwise to the solution of clopidogrel (free base) dissolved in water or organic solvent. More specifically, (+)-clopidogrel isomer is reacted with the cation exchange resin in liquid state. Both solutions or their mixture thereof allow to stand at room temperature. It is preferred to maintain a cooling state for a high yield of the solid-form clopidogrel. For a higher yield of the solid-form clopidogrel the supernatant is removed after precipitation of the solid form, and then the same amount of anhydrous organic solvent as removed amount is added to the solution and allows to stand. The solid precipitate thus formed is washed with an organic solvent and filtered off. The organic solvent used for the methodology of the present invention is employed at room temperature or preferably, at a cooling state.

When clopidogrel salts are produced in the presence of the well known inorganic or organic solvents, a desired product cannot be precipitated, or it is suspended or emulsified, even in the presence of a small amount of water in a solvent system where clopidogrel (free base) is reacted with some acids. In contrast, the solid form according to the prevent invention may be obtained in higher yield even in the small amount of water. The final solid precipitate is evaporated under reduced pressure to dryness to obtain the clopidogrel-resinate complex in powder form that may provide excellent flowability.

Additionally, except for sulfonic acid groups to be contacted with clopidogrel salts in the process of preparing the clopidogrel-resinate complex, the remaining sulfonic acid group may be masked by an alkali material. Alkali materials according to the present invention include alkali metals, alkaline earth metals and amines. Alkali materials is one or more selected from alkali metal materials or alkaline earth metal materials (sodium hydroxide, potassium hydroxide, sodium methoxide, potassium methoxide, sodium ethoxide and potassium ethoxide) and amines (ammonia, methylamine, ethylamine, ethanolamine, alginine and histidine), but not limited to the aforementioned alkali materials.

The clopidogrel-resinate complex according to the present invention is obtained such that (+)-clopidogrel isomer (free base) and cation exchange resin is mixed in a weight ratio of 1:0.1 to 1:10, preferably 1:0.2 to 1:5, and more preferably 1:0.5 to 1:2.

Further, the clopidogrel-resinate complex of the present invention provides excellent flowability to its powder form with an angle of repose of about 30° to 40°. Thus its large-scale industrial production with higher yield of solid-form product may be expected.

Additionally, there is a stability problem which is apparent in the general clopidogrel-resinate complex composed of water-insoluble ion exchange resins in the art. In contrast, the water-soluble ion exchange resin based clopidogrel-resinate complex has better stability, which cannot be achieved by the water-insoluble ion exchange resin.

As the water-insoluble ion exchange resin that is bounded to clopidogrel (free base) cannot be precipitated in a structure where two molecules are regularly arranged, the use of higher amounts of water-insoluble ion exchange resin should be employed for gaining the solid material. If large excess amounts of water-insoluble ion exchange resin is used for a higher yield of the solid product, no better stability will be gained than oily (+)-clopidogrel isomer (free base) itself. In contrast, the clopidogrel-resinate complex of the present invention, so formed by using a water-soluble ion exchange resin, has unexpectedly better stability in the chemical structure of clopidogrel. This is meant that the final product of the present invention may maintain its stability until its expiry date, since the occurrence of any impurities associated with clopidogrel is minimal during the storage period.

A composition comprising the drug-resin complexes of the present invention surprisingly has taste-masking capabilities (strong irritation, bitterness and obnoxious taste) against(+)-clopidogrel sulfate. Clopidogrel hydrogen sulfate is a drug which is unpleasant to take orally in the mouth and/or gastrointestinal tract due to a strong irritation, bitterness and sour taste. In contrast, clopidogrel-resinate complex of the present invention has a masking effect against such irritation, bitterness and sour tastes in the mucosa, with very mild sour but pleasant tasting, when taking orally.

The clopidogrel-resinate complex of the present invention rapidly melts in the mouth without impurities, while the conventional water-insoluble ion exchange resin does not melt in the mouth and impurities remain.

Some examples of drugs in which the taste masking technique has been successfully demonstrated, those skilled in the art may appreciate that the scope of this approach is limited with little effects in pharmacology.

Unlike the general drug-ion exchange resin preparations of the prior art, therefore, the clopidogrel-resinate complex of the present invention has been unexpectedly found to address the problem of obnoxious and sour taste, when taking clopidogrel orally. As a result, the clopidogrel-resinate complex may improve patient compliance, while it requires no drink of water during administration and can be formulated into a liquid form.

The (+)-clopidogrel isomer resinate complex according to the present invention, so formed, may be formulated into a variety of dosage forms using a pharmaceutically acceptable common technique such as blending, kneading, grinding, sieving, filling, compressing, lyophilization, spray-drying, fluid-bed drying and centrifugal granulation.

The pharmaceutical composition of the present invention may be formulated using conventional, pharmaceutically acceptable excipients. Such conventional, pharmaceutically acceptable excipients include diluents, binders, disintegrants, coloring agents, sweetening agents, flavors, preservatives, lubricants and a mixture thereof.

Suitable one or more diluents include, but not limited to one or more selected from a group consisting of lactose, dextrose, microcrystalline cellulose and starch, but not limited to the aforementioned the diluents.

Suitable one or more binders include, but not limited to polyvinyl pyrrolidone, hydroxypropyl cellulose, hyrdoxypropylmethyl cellulose, hydroxyethyl cellulose, dicalcium phosphate and sodium alginate.

Suitable one or more disintegrants include, but not limited to croscarmellose sodium, sodium starch glycolate, cross-linked polyvinyl pyrrolidone, starch paste, low-substituted hydroxypropyl cellulose.

Suitable one or more coloring agents include, but not limited to soluble and tar pigments.

Suitable one or more sweetening agents include, but not limited to dextrose, sorbitol, mannitol, aspartame, acesulfame and citric acid.

Suitable one or more flavors include, but not limited to orange flavor powder, grape flavor powder, strawberry flavor powder and blueberry flavor powder.

Suitable one or more preservatives include, but not limited to benzoic acid, methylparabene, ethylparabene and propylparabene.

Suitable one or more lubricants include, but not limited to magnesium stearate, talc, hard silicon dioxide and sucrose fatty acid ester.

### ADVANTAGEOUS EFFECTS

In one aspect, the present invention provides a novel resinate complex of (+)-clopidogrel isomer with high purity without any impurities encountered in the conventional process of using organic or inorganic salts.

In another aspect, the present invention provides the novel resinate complex of (+)-clopidogrel isomer with better stability in a chemical structure, thus improving its formulation stability.

In another aspect, the present invention allows an easier formulation of the resinate complex of (+)-clopidogrel isomer into a variety of dosage forms with no additional process, as it removes the hygroscopicity of crystallized clopidogrel and provides excellent flowability to a powder form.

In another aspect, the present invention provides the novel resinate complex of (+)-clopidogrel isomer that requires no drink of water with patient compliance by taste masking of a bitter drug, and its liquid form may be formulated.

### BRIEF DESCRIPTION OF THE DRAWINGS

FIG. 1 shows the results of stress testing on the samples produced by Examples 1, 2 and 8, including Comparative examples 1 and 2.

### BEST MODE FOR CARRYING OUT THE INVENTION

This invention will now be described by reference to the following examples and experimental examples which are merely illustrative and which are not to be construed as a limitation of the scope of this invention.

### EXAMPLE 1

### Methyl(+)-(S)-(2-chlorophenyl)(6,7-dihydro-4H-thieno[3,2-c]pyridine-5-yl)acetate-styrene sulfonate polymer complex

2.40 g (7.45 mmole) of (+)-clopidogrel isomer (free base) was dissolved in 50 mL of acetone, cooled and stirred. 1.25 mL of 18% styrene sulfonate polymer solution was slowly added to the cooled solution. The resulting solution was stirred for some time, during which a precipitate was formed. After a supernatant of the solution was decanted, 20 mL of cooled acetone was added and stirred. Then, a solid precipitation was collected by filtration and dried in a vacuum oven. About 54.2% of (+)-clopidogrel isomer to a total dried mass was contained in the collected resinate complex, which was identified by HPLC analysis.

### EXAMPLE 2

### Methyl(+)-(S)-(2-chlorophenyl)(6,7-dihydro-4H-thieno[3,2-c]pyridine-5-yl)acetate-styrene sulfonate polymer complex

0.44 g (1.35 mmole) of (+)-clopidogrel isomer (free base) was dissolved in 50 mL of acetone, cooled and stirred. 1.2 mL of 18% styrene sulfonate polymer solution was slowly added to the cooled solution. The resulting solution was stirred for some time, during which a precipitate was formed. After a supernatant of the solution was decanted, 20 mL of cooled acetone was added and stirred. Then, a solid precipitation was collected by filtration and dried in a vacuum oven. About 52.1% of (+)-clopidogrel isomer to a total dried mass was contained in the collected resinate complex, which was identified by HPLC analysis.

### EXAMPLE 3

### Methyl(+)-(S)-(2-chlorophenyl)(6,7-dihydro-4H-thieno[3,2-c]pyridine-5-yl)acetate-styrene sulfonate polymer complex

2.40 g (7.45 mmole) of (+)-clopidogrel isomer (free base) was dissolved in 100 mL of acetone, cooled and stirred. 1.2 mL of 18% styrene sulfonate polymer solution was slowly added to the cooled solution. The resulting solution was stirred for some time, during which a precipitate was formed. After a supernatant of the solution was decanted, 50 mL of cooled acetone was added and stirred. Then, a solid precipitation was collected by filtration and dried in a vacuum oven. About 53.6% of (+)-clopidogrel isomer to a total dried mass was contained in the collected resinate complex, which was identified by HPLC analysis.

### EXAMPLE 4

### Methyl(+)-(S)-(2-chlorophenyl)(6,7-dihydro-4H-thieno[3,2-c]pyridine-5-yl)acetate-styrene sulfonate polymer complex

0.44 g (1.35 mmole) of (+)-clopidogrel isomer (free base) was dissolved in 100 mL of acetone, cooled and stirred. 1.2 mL of 18% styrene sulfonate polymer solution was slowly added to the cooled solution. The resulting solution was stirred for some time, during which a precipitate was formed. After a supernatant of the solution was decanted, 50 mL of cooled acetone was added and stirred. Then, a solid precipitation was collected by filtration and dried in a vacuum oven. About 50.7% of (+)-clopidogrel isomer to a total dried mass was contained in the collected resinate complex, which was identified by HPLC analysis.

### EXAMPLE 5

### Methyl(+)-(S)-(2-chlorophenyl)(6,7-dihydro-4H-thieno[3,2-c]pyridine-5-yl)acetate-styrene sulfonate polymer complex

2.61 g (8.13 mmole) of (+)-clopidogrel isomer (free base) was dissolved in 100 mL of diethylether, cooled and stirred. 2.5 mL of 18% styrene sulfonate polymer solution was slowly added to the cooled solution. The resulting solution was stirred for some time, during which a precipitate was formed. After a supernatant of the solution was decanted, 50 mL of cooled acetone was added and stirred. Then, a solid precipitation was collected by filtration and dried in a vacuum oven. About 49.1% of (+)-clopidogrel isomer to a total dried mass was contained in the collected resinate complex, which was identified by HPLC analysis.

### EXAMPLE 6

### Methyl(+)-(S)-(2-chlorophenyl)(6,7-dihydro-4H-thieno[3,2-c]pyridine-5-yl)acetate-styrene sulfonate polymer complex

0.87 g (2.71 mmole) of (+)-clopidogrel isomer (free base) was dissolved in 50 mL of diethylether, cooled and stirred. 2.5 mL of 18% styrene sulfonate polymer solution was slowly added to the cooled solution. The resulting solution was stirred for some time, during which a precipitate was formed. After a supernatant of the solution was decanted, 50 mL of cooled acetone was added and stirred. Then, a solid precipitation was collected by filtration and dried in a vacuum oven. About 47.8% of (+)-clopidogrel isomer to a total dried mass was contained in the collected resinate complex, which was identified by HPLC analysis.

### EXAMPLE 7

### Methyl(+)-(S)-(2-chlorophenyl)(6,7-dihydro-4H-thieno[3,2-c]pyridine-5-yl)acetate-styrene sulfonate polymer complex

2.40 g (7.45 mmole) of (+)-clopidogrel isomer (free base) was dissolved in 36 mL of acetone and 36 mL of ethanol and cooled. The cooled solution was stirred and with the slow addition of 9.0 g (9.77 mmol) of 20 wt % styrene sulfonate polymer in ethanol solution, stirred. After stirring, the solution allowed to stand for some time, during which a precipitate was formed. After a supernatant of the solution was decanted, 50 mL of cooled t-butylmethylether was added, stirred and filtered off. Then, 50 mL of cooled ethanol was added to a solid precipitation, stirred. The solid precipitation was collected by filtration and dried in a vacuum oven. About 52.1% of (+)-clopidogrel isomer to a total dried mass was contained in the collected resinate complex, which was identified by HPLC analysis.

### EXAMPLE 8

### Methyl(+)-(S)-(2-chlorophenyl)(6,7-dihydro-4H-thieno[3,2-c]pyridine-5-yl)acetate-styrene sulfonate polymer complex

2.40 g (7.45 mmole) of (+)-clopidogrel isomer (free base) was dissolved in 36 mL of acetone and 36 mL of ethanol and cooled. The cooled solution was stirred and with the slow addition of 9.0 g (9.77 mmol) of 20 wt % styrene sulfonate polymer in ethanol solution, stirred. Then, the resulting solution was stirred, followed by the addition of 0.11 g (1.85 mmol) of sodium methoxide plus ethanol (0.25 g/mL) solution. After stirring, the solution allowed to stand for some time, during which a precipitate was formed. After a supernatant of the solution was decanted, 50 mL of cooled t-butylmethylether was added, stirred and filtered off. Then, 50 mL of cooled ethanol was added to a solid precipitation, stirred. The solid precipitation was collected by filtration and dried in a vacuum oven. About 51.6% of (+)-clopidogrel isomer to a total dried mass was contained in the collected resinate complex, which was identified by HPLC analysis.

### EXAMPLE 9

### Methyl(+)-(S)-(2-chlorophenyl)(6,7-dihydro-4H-thieno[3,2-c]pyridine-5-yl)acetate-styrene sulfonate polymer complex

2.40 g (7.45 mmole) of (+)-clopidogrel isomer (free base) was dissolved in 36 mL of acetone and 36 mL of ethanol and cooled. The cooled solution was stirred and with the slow addition of 9.0 g (9.77 mmol) of 20 wt % styrene sulfonate polymer in ethanol solution, stirred. Then, the resulting solution was stirred, followed by the addition of 0.11 g (1.85 mmol) of sodium methoxide plus ethanol (0.25 g/mL) solution. After stirring, the solution allowed to stand for some time, during which a precipitate was formed. After a supernatant of the solution was decanted, 50 mL of cooled iso propylether was added, stirred and filtered off. Then, 50 mL of cooled ethanol was added to a solid precipitation, stirred. The solid precipitation was collected by filtration and dried in a vacuum oven. About 51.8% of (+)-clopidogrel isomer to a total dried mass was contained in the collected resinate complex, which was identified by HPLC analysis.

### EXAMPLE 10

### Formulation of Clopidogrel-Styrene Sulfonate Polymer Complex

15.0 g of (+)-clopidogrel isomer-resinate complex, so formed from Example 8, was blended with a mixture containing 2.0 g of sodium starch glycolate as a disintegrant, 1.0 g of glyceryl behenate as a lubricant, and a proper amount of microcrystalline cellulose such that a total weight may be 35.0 g. Using a tabletting machine, the final product mixture was used to prepare a tablet such that its active ingredient may contain 75 mg of clopidogrel (free base).

### EXAMPLE 11

### Formulation of Clopidogrel-Styrene Sulfonate Polymer Complex

15.0 g of (+)-clopidogrel isomer-resinate complex, so formed from Example 2, was blended with 6.0 g of microcrystalline cellulose as a diluent. The mixture was pulverized by compaction and filtered by a 18-mesh sieve to form a granule. To the granule were added 4.0 g of sodium starch glycolate as a disintegrant, 1.0 g of glyceryl behenate as a lubricant, and a proper amount of microcrystalline cellulose such that a total weight may be 35.0 g. Using a tabletting machine, the final mixture was used to prepare a tablet such that its active ingredient may contain 75 mg of clopidogrel (free base).

### EXAMPLE 12

### Formulation of Clopidogrel-Styrene Sulfonate Polymer Complex

15.0 g of (+)-clopidogrel isomer-resinate complex, so formed from Example 2, was blended with 1.0 g of glyceryl behenate as a lubricant, 3.0 g of povidone as a binder, and 13.0 g of microcrystalline cellulose as a diluent. The mixture was pulverized by compaction and filtered by a 18-mesh sieve to form a granule. To the granule were added 2.0 g of sodium starch glycolate as a disintegrant, and 1.0 g of glyceryl behenate as a lubricant. Using a capsule filling machine, the final mixture was subject to moderate pressure to form a slurry into which a capsule was filled.

### COMPARATIVE EXAMPLE 1

### Methyl(+)-(S)-(2-chlorophenyl)(6,7-dihydro-4H-thieno[3,2-c]pyridine-5-yl)acetate-Amberlite complex

1 g of a water-insoluble cation exchange resin Amberlite IRP-69 (activating sulfonic acid group via deionization of metal ions) was added to 0.44 g (1.35 mmole) of (+)-clopidogrel isomer (free base) in 2 g of ethanol and stirred for about 60 minutes.

Then the solvent was evaporated to dryness under reduced pressure and with the addition of 1 g of hexane, the resulting solution was stirred and re-dried. The dried product was washed with a cooled hexane and dried in a vacuum oven. About 15.4% of (+)-clopidogrel isomer to a total dried mass was contained in the collected resinate complex, which was identified by HPLC analysis.

### COMPARATIVE EXAMPLE 2

### Methyl(+)-(S)-(2-chlorophenyl)(6,7-dihydro-4H-thieno[3,2-c]pyridine-5-yl)acetate-Amberlite complex

1 g of a water-insoluble cation exchange resin Amberlite IRP-69 with carboxylic acid was added to 0.44 g (1.35 mmole) of (+)-clopidogrel isomer (free base) in 2 g of ethanol and stirred for about 60 minutes. Then the solvent was evaporated to dryness under reduced pressure and with the addition of 1 g of hexane, the resulting solution was stirred and re-dried. The dried product was washed with a cold water and dried in a vacuum oven. About 6.2% of (+)-clopidogrel isomer to a total dried mass was contained in the collected resinate complex, which was identified by HPLC analysis.

### EXPERIMENTAL EXAMPLE 1

### Chemical Structure and Melting Points

The chemical structure of (+)-clopidogrel isomer-resinate complex, so obtained from Examples 8 and 9, was determined using MeOH-d as a solvent, as shown below.

¹H-NMR: 1.20-1.90 (m, 5H), 3.13 (m, 2H), 3.73 (m, 5H), 4.25 (m, 2H), 5.81 (s, 1H), 6.30-6.75 (m, 4H), 7.28 (d, J=4.05 Hz, 1H), 7.44-7.60 (m, 6H), 7.70(d, J=7.28 Hz, 1H).

¹³C-NMR: 19.39, 40.35, 48.01, 50.69, 53.51, 65.53, 123.57, 125.02, 125.31, 125.85, 126.58, 126.77, 127.42, 128.61, 130.75, 131.40, 132.66, 135.14, 142.43, 147.38, 166.91.

Also, the (+)-clopidogrel optical isomer-resinate complex was decomposed at 225-235° C. using a melting point device.

### EXPERIMENTAL EXAMPLE 2

### Heat Stability Test

Each of the (+)-clopidogrel isomer-resinate complex, so obtained from Examples 1, 2 and 8, including a solid form of (+)-clopidogrel optical isomer-Amberlite complex and (+)-clopidogrel isomer (free base), so obtained from Comparative examples 1 and 2, were put into a tightly closed container at 80° C. to determine the contents of clopidogrel in the baseline, day 1 and day 3 by HPLC analysis in accordance with a content uniformity test of the U.S. Pharmacopoeia.

(HPLC Analysis)
Column: ULTRON ES-OVM chiral 5 [symbol] , 4.6×250 mm
Mobile phase: 0.01 M potassium dihydrogen phosphate solution/acetonitrile 75:25
Flow rate: 1 mL/min
Column temperature: Room temperature
Detector: Ultraviolet absorption spectrophotometer (detection wavelength: 220 nm)
Injection volume: 10

[symbol]

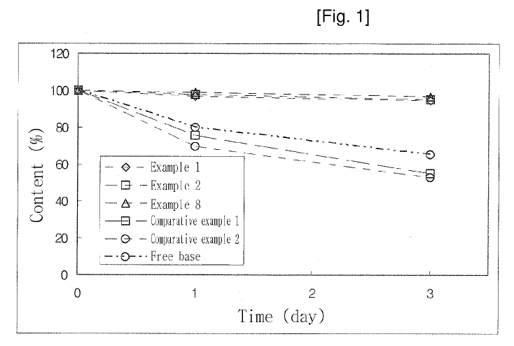

As shown in FIG. 1, no significant decrease in content and/or change in appearance was observed from the clopidogrel-resinate complex of the present invention under the stress storage conditions of 80° C., demonstrating its better stability profile.

The oily (+)-clopidogrel isomer (free base) itself was subject to rapid degradation under the stress conditions, showing 70% decrease in content at day 3. Also, the solid form of (+)-clopidogrel optical isomer-Amberlite complex, so obtained from Comparative examples 1 and 2, showed a rapid degradation of clopidogrel with less than 60% in content at day 3. It is well understood that molecules even in the solid form of clopidogrel bonded with a common ion exchange resin are in extremely unstable state.

### EXPERIMENTAL EXAMPLE 3

### Comparison of Taste and Mucosal Irritation in Sensory Test

A sensory test of five volunteers was conducted to compare the taste and mucosal irritation of (+)-clopidogrel optical isomer-styrene sulfonate complex (75 mg as free base), so obtained from Examples 1, 2 and 9, with commercially-available clopidogrel hydrogen sulfate (75 mg as free base), which were prepared in a solid form containing no excipient, water or solvent. The results were shown in Table 1.

As noted in Table 1, all subjects had a bitter and obnoxious taste in the commercially-available clopidogrel hydrogen sulfate and thus the sensory test had to discontinue one minute after tasting the drug. By contrast, the resinate complex of the present invention (Examples 1, 2 and 9) did not induce irritation or bitterness in the mouth but with some mild sour taste or no taste.

As the clopidogrel-resinate complex of the present invention provides excellent taste-masking capabilities, patients may be able to take it without a drink of water, thus contributing to patient's compliance. Also, the complex may be formulated into a liquid form.

TABLE 1

| | Irritation | Bitterness | Sour taste |
|---|---|---|---|
| Example 1 | No | No | Very mild |
| Example 2 | No | No | Very mild |
| Example 9 | No | No | Little taste |
| Clopidogrel | Very strong | Sustained | Very severe |
| hydrogen sulfate | | bitterness after | |
| | | irritation | |

1. A resinate complex of (+)-clopidogrel isomer, wherein a (+)-clopidogrel isomer is bound to a water-soluble cation exchange resin having sulfonic acid groups.

2. The resinate complex of (+)-clopidogrel isomer according to claim 1, wherein the water-soluble cation exchange resin having sulfonic acid groups is bounded to the (+)-clopidogrel isomer in the ratio of 1:0.1 to 1:10 by weight.

3. The resinate complex of (+)-clopidogrel isomer according to claim 1, wherein one or more water-soluble cation exchange resin having sulfonic acid groups are selected from cation exchange resins having a molecular weight range of 5,000 to 1,000,000.

4. The resinate complex of (+)-clopidogrel isomer according to claim 1, wherein one or more water-soluble cation exchange resin having sulfonic acid groups are selected from a group consisting of styrene sulfonate polymer and divinylbenzene styrene sulfonate copolymer.

5. The resinate complex of (+)-clopidogrel isomer according to claim 4, wherein the water-soluble cation exchange resin having sulfonic acid groups has one or more polymers selected from styrene sulfonate polymer.

6. The resinate complex of (+)-clopidogrel isomer according to claim 1, wherein said sulfonic acid groups remaining in the resinate complex of (+)-clopidogrel isomer is masked by an alkali material.

7. The resinate complex of (+)-clopidogrel isomer according to claim 6, wherein one or more alkali material are selected from a group consisting of alkali metal and earth metal, and amine.

8. A solid pharmaceutical composition containing said resinate complex of (+)-clopidogrel isomer as an active ingredient, wherein a (+)-clopidogrel isomer is bounded to a water-soluble cation exchange resin having sulfonic acid groups.

9. The solid pharmaceutical composition according to claim 8, wherein the water-soluble cation exchange resin having sulfonic acid groups is bounded to the (+)-clopidogrel isomer in the ratio of 1:0.1 to 1:10 by weight.

10. A process for manufacturing the resinate complex of (+)-clopidogrel isomer, wherein the water-soluble cation exchange resin having sulfonic acid groups is bounded to the (+)-clopidogrel isomer in aqueous state.
